(19) United States
(12) Patent Application Publication
(10) Pub. No.: US 2004/0146536 A1
(43) Pub. Date: Jul. 29, 2004

(54) Styptic pencil or stick housed in a tubular dispenser

(76) Inventor: Jeffrey L. Kaplan, Fort Lauderdale, FL (US)

Correspondence Address: JEFFREY L. KAPLAN, POB 11106, Ft. Lauderdale, FL, 33339, US

(21) Appl. No.: 10/350,943

(22) Filed: Jan. 27, 2003

(51) Int. Cl. A61K 9/00

(52) U.S. Cl. 424/400

(57) ABSTRACT

A styptic pencil or stick composition storage dispensing unit comprising a tubular housing, said housing providing an interior dry space for a styptic pencil or stick composition, an attached or removable cap or cover, an ejector for controllably advancing or ejecting said styptic pencil or stick composition through said housing by mechanical rotation or mechanical force without rotation.

### BACKGROUND

1. Field of Invention

This invention relates to a styptic pencil or stick dispenser comprising a styptic composition in stick or pencil form and a tube or sleeve which houses the styptic material and through which the styptic material is advanced and used to curtail bleeding from minor nicks and cuts.

2. Discussion of Prior Art

Nicks and cuts on the chin, face and other areas are a virtually inevitable consequence of regular blade shaving. Nicks and cuts can also be a significant problem for women when shaving their legs and underarms. Shaving cuts, especially in the area of the face tend to bleed profusely, and it can be quite difficult to halt the flow of blood.

The use of styptic or astringent compositions is well established in the art. The principal astringent chemicals are compounds of aluminum, potassium, zinc, manganese, iron, bismuth and other chemical groups that contain these metals (such as permanganates). Although some astringent substances are potent chemicals, they do not enter human body cells, which is their virtue. Rather, all their activity occurs at cell surface where they block noxious substances from entering the cell or the space between cells. They coagulate body chemicals so that they form clots, crusts or other solid deposits.

To go back to the original example of the man who cuts himself while shaving, an astringent styptic cast pencil is normally used. These styptic pencils have been in used since 1890 and usually consist of 80-90% aluminum or potassium sulfate and 10-20% of an inert filler. Currently, the only type of styptic compositions available to the public are the generic molded cast solid styptic pencil, a stick design or a powdered styptic composition.

Typical styptic compositions in pencil and stick forms are set forth in U.S. Pat. No. 5,718,865 to Askew (1998), in U.S. Pat. No. 819,901 to Maschal (1906), in U.S. Pat. No. 459,738 to Black (1890) and in U.S. Pat. No. 497,659 to Harned (1892). These cited examples all show various improved ways dealing with the manufacture and production procedures of making styptic pencils or sticks but do not deal with the problems associated with storing and preserving said styptic pencils or sticks when used by the general public.

Manufacturing Processes:

Currently, the generic solid molded styptic pencil or stick designs are manufactured using conventional techniques such as mechanical means, nozzle filling or flood filling and then simply placed in a flimsy covered plastic vial. Using mechanical means, solid pieces of alum are cut and grinded into pencil or stick shapes. In the nozzle fill method a homogeneous styptic composition is dispensed from a filling nozzle directly into molds. In the flood fill method the molds are placed in an opening of a tray and the styptic composition is poured into the tray thereby flooding the tray and in turn filling the molds.

Consumers Usage:

When needed, the user removes the entire exposed pencil or stick from the plastic vial and applies water to the tip edge, which in turn softens the styptic composition. Once the tip is wet, the user applies the styptic composition to the wound. Since the user is normally shaving and is in the bathroom, the entire exposed styptic pencil or stick gets wet from the users hands. After use, the entire exposed styptic pencil or stick is supposed to be dried and placed back into the plastic vial for future use. Unfortunately, when the entire exposed styptic pencil or stick is repeatedly wetted and stored, the entire exposed pencil or stick begins to crumble, deteriorate and normally adheres to the inside of the plastic vial container thus causing further damage when removal is attempted

Even if the average user dries the styptic pencil or stick properly, within a few weeks of normal use the entire styptic pencil or stick becomes so deteriorated it must be thrown away.

In various attempts to obviate these problems, several different types of styptic devices have been introduced. These consist of styptic bandages in U.S. Pat. No. 3,113,568 to Robins (1963), in U.S. Pat. No. 4,022,203 to Ackley (1977), and in U.S. Pat. No. 4,233,976 to Dunshee (1980).

In addition, styptic creams have been introduced in U.S. Pat. No. 4,166,108 and in U.S. Pat. No. 5,279,837.

However, none of these unique styptic devices have been accepted by the mass public. Thus, a need exists for a styptic pencil or stick dispenser comprising a styptic pencil or stick and a tube or sleeve which houses the styptic pencil or stick through which the styptic pencil or stick is advanced or ejected.

### OBJECTS AND ADVANTAGES

Accordingly, several objects and advantages of my invention are:

a) To provide a novel styptic pencil or stick dispenser.

b) To provide a novel styptic pencil or stick dispenser comprising a styptic pencil or stick composition and a tube or sleeve which houses the styptic pencil or stick composition and through which the styptic pencil or stick composition is advanced.

c) To provide a novel styptic pencil or stick dispenser which will keep the enclosed styptic composition stable and free from water and moisture.

d) To provide a novel styptic pencil or stick dispenser which is simple, inexpensive and easy to use.

### DETAILED DESCRIPTION OF THE INVENTION

In the present invention, the foregoing difficulties are obviated in that there is provided a styptic composition housed in a tubular dispenser thereby keeping water and moisture from the styptic composition until ready to use by advancing or ejecting said styptic composition from the tubular dispenser.

Styptic Pencil or Stick Dispenser:

The manufacturing process of a simple plastic tubular dispenser is well established in the art using conventional techniques such as injection molding and the like. It is anticipated that the styptic pencil or stick composition will be manufactured directly into the container in which it is sold, so that the container also serves as a mold during the casting process, to wit; a lip balm, sun screen, deodorant or glue stick type dispenser with a moisture-locking cap and movable base. The base advances or ejects the styptic pencil or stick composition through the dispenser such as by mechanical rotation or mechanical force without rotation onto the users face or other area where the bleeding has occurred.

Styptic Pencil or Stick Composition:

The term “styptic pencil or stick composition” is defined as a solid or semi-solid malleable styptic composition that is sufficiently solid so as to substantially retain its shape when advanced or ejected from an open end of the container, while capable of leaving a desired residue when applied by wetting.

The styptic pencil or stick composition is well established in the art comprising ingredients such as aluminum sulfate, potassium sulfate, zinc, manganese, iron, titanium dioxide, bismuth and other chemical groups that contain these metals (such as permanganates).

Well-known manufacturing processes such as nozzle filling or flood filling may be utilized. In addition by adding plasticisers and other additives, fragrances, skin conditioning agents, moisturizers and the like can be included in the manufacturing process.

### CONCLUSION, RAMIFICATION AND SCOPE OF THE INVENTION

Accordingly, the reader will see that a styptic pencil or stick composition housed in a tubular dispenser of the inventor provides that:

It provides an inexpensive solution to the brittle, drying, deteriorating and sticking properties associated with current exposed styptic pencils or sticks available to the public.

Those skilled in the art will have no difficulty in determining suitable proportions of the above agents to be used. The invention has been described as applied to preferred embodiments and it will be understood that various substitutions and changes may be effected without departing from the spirit and scope of the novel concepts and principals of this invention.

What is claimed is:

1. A styptic pencil or stick composition storage and dispensing unit comprising:
a tubular housing, said housing providing an interior space for a styptic pencil or stick composition, said housing providing an interior base defining a bottom ejector, said bottom ejector communicating with said interior styptic pencil or stick composition;
a cover or cap, said cover or cap attached or removable to said housing, covering and exposing the styptic pencil or stick composition;
an ejector for controllably advancing or ejecting the styptic pencil or stick composition through said housing such as by mechanical rotation or mechanical force without rotation.

2. The styptic pencil or stick composition storage and dispensing unit as set forth in claim 1, wherein the styptic pencil or stick composition is substantially wholly contained inside said housing and is filled by conventional manufacturing processes such as mechanical means, nozzle filling, flood filling or other known manufacturing methods.

3. The styptic pencil or stick composition storage and dispensing unit as set forth in claim 1, wherein the housing unit resembles tubular lip balms, sun-screens deodorants or glue stick designs.

4. The styptic pencil or stick composition storage and dispensing unit as set forth in claim 3, wherein the housing unit is designed and inexpensively manufactured to keep said styptic pencil or stick styptic composition dry, free from water and moisture.

5. The styptic pencil or stick composition storage and dispensing unit as set forth in claim 1, wherein the styptic pencil or stick composition contained in the housing is manufactured using astringent chemicals or natural ingredients which coagulate blood, such as alum, aluminum sulfate, aluminum acetate, potassium sulfate, potassium alum, ammonium alum, potassium nitrate, aluminum chloride, ammonium chloride, hexahydrate, titanium dioxide, zinc sulfate, manganese, iron alum, caesium alum, chrome alum, chromoselenic alum, bismuth, copper sulfate, iron sulfate, tannin acid and other chemical or natural organic groups that contain these metals such as permanganates.

6. The styptic pencil or stick composition as defined in claim 5, wherein the styptic pencil or stick composition is a solid or semi solid malleable styptic composition which retains its shape when advanced or ejected from an open end of the tubular housing, while capable of leaving a desired residue when applied by wetting.

7. The styptic pencil or stick composition as defined in claim 5, wherein the styptic pencil or styptic composition contains additional ingredients such as fragrances, skin conditioning agents, moisturizers, preservatives, emollients, surfactants, medicaments, colorants, flavorants, perfumes, polyol plasticisers, glycerol, sorbitol, borax, antiseptic compounds, hamamelis water NF X1 (witch hazel), aloe extracts, vitamins, plant/herb derivatives and water.
